(19) United States
(12) Patent Application Publication
(10) Pub. No.: US 2024/0060070 A1
(43) Pub. Date: Feb. 22, 2024

(54) TREATMENT OF CANCER ASSOCIATED WITH VARIANT NOVEL OPEN READING FRAMES

(71) Applicants: Cambridge Enterprise Limited, Cambridge (GB); International Centre for Genetic Engineering and Biotechnology, Delhi (IN)

(72) Inventor: Sudhakaran PRABAKARAN, Acton, MA (US)

(21) Appl. No.: 18/267,223

(22) PCT Filed: Dec. 15, 2021
(86) PCT No.: PCT/IB2021/061804

§ 371 Date: Jun. 14, 2023

Related U.S. Application Data
(60) Provisional application No. 63/126,371, filed on Dec. 16, 2020.

(51) Int. Cl. C12N 15/113; A61K 31/4155; A61K 31/4709; C07K 14/82; C12N 15/86

(52) U.S. Cl. CPC C12N 15/1135; A61K 31/4155; A61K 31/4709; C07K 14/82; C12N 15/86; C12N 2310/11; C12N 2740/15043

(57) ABSTRACT

The present application features methods of treating a cancer associated with a genetic variant. The genetic variant is also present within a novel open reading frame (nORF) associated with the gene in which the variant leads to increased or reduced expression of the variant nORF.

### CROSS-REFERENCE TO RELATED APPLICATIONS

This application claims the benefit of U.S. Provisional Application No. 63/126,371 filed on Dec. 16, 2020, which is incorporated herein by reference in its entirety.

### BACKGROUND OF THE INVENTION

Many cancers are caused by genetic mutations that present, at face, to be benign to the gene or the canonical protein encoded by the gene that is associated with the cancer. Thus, it is unclear how certain benign genetic variants contribute to cancer pathology under these circumstances. Furthermore, as it is unclear how these mutations contribute to the underlying cancer pathology, providing an effective therapeutic remains a challenging endeavor. Accordingly, new methods of diagnosis and treatment are needed to better understand how these benign variants cause a wide range of cancers.

### SUMMARY OF THE INVENTION

In one aspect, the invention features a method of treating a cancer in a subject by identifying a sequence variant of a novel open reading frame (nORF) and a cancer associated therewith, wherein the sequence of the nORF is distinct from a canonical open reading frame (cORF) of a gene. The nORF is present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene, and wherein the variant nORF has increased expression relative to the nORF. The method further includes administering to the subject an inhibitor that reduces expression of the variant nORF to treat the cancer.

In another aspect, the invention features a method of treating a cancer in a subject by administering to the subject an inhibitor that reduces expression of a variant nORF. The subject may have previously been identified with a sequence variant of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has increased expression relative to the nORF.

In some embodiments of either of the foregoing aspects, the method reduces expression of the nORF, e.g., by at least 5%, 10%, 15%, 20%, 25%, 30%, 40%, 50%, 60%, 70%, 80%, 90%, 95%, 97%, or 99%. The nORF may exhibit an increase (e.g. by at least 5%, 10%, 15%, 20%, 25%, 30%, 40%, 50%, 60%, 70%, 80%, 90%, 100%, 200%, 300%, 400%, 500%, or more) in expression, e.g., as compared to the WT nORF or as compared to the variant nORF in normal (e.g., noncancerous) tissue.

In some embodiments of either of the above aspects, the inhibitor is a small molecule, a polynucleotide, or a polypeptide. The polynucleotide may include, e.g., a miRNA, an antisense RNA, an shRNA, or an shRNA. The polypeptide may include, e.g., an antibody or antigen-binding fragment thereof (e.g., an scFv).

In some embodiments, the inhibitor is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include, e.g., one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be, e.g., a lentiviral vector.

In some embodiments, the pseudotyped viral vector includes one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the pseudotyped viral vector includes a VSV-G envelope protein.

In another aspect, the invention features a method of treating a cancer in a subject by identifying a sequence variant of a nORF and a cancer associated therewith, wherein the sequence of the nORF is distinct from a cORF of a gene. The nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has decreased expression relative to the nORF. The method further includes administering to the subject an activator that increases expression of variant nORF to treat the cancer.

In another aspect, the invention features a method of treating a cancer in a subject by administering to the subject an activator that increases expression of a variant nORF. The subject may have previously been identified with a sequence variant of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has decreased expression relative to the nORF.

In some embodiments of either of the foregoing aspects, the method increases expression of the nORF, e.g., by at least 5%, 10%, 15%, 20%, 25%, 30%, 40%, 50%, 60%, 70%, 80%, 90%, 100%, 200%, 300%, 400%, 500%, or more. The nORF may exhibit a decrease (e.g., by at least 5%, 10%, 15%, 20%, 25%, 30%, 40%, 50%, 60%, 70%, 80%, 90%, 95%, 97%, or 99%) in expression, e.g., as compared to the WT nORF or as compared to the variant nORF in normal (e.g., noncancerous) tissue.

In some embodiments, the activator is a small molecule, a polynucleotide, or a polypeptide. The polynucleotide may include, e.g., an antisense RNA. The polypeptide may include, e.g., an antibody or antigen-binding fragment thereof (e.g., an scFv).

In some embodiments, the activator is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an AAV vector.

In some embodiments, the viral vector is a Retroviridae family viral vector (e.g., a lentiviral vector, an alpharetroviral vector, or a gammaretroviral vector). The Retroviridae family viral vector may include, e.g., one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be, e.g., a lentiviral vector.

In some embodiments, the pseudotyped viral vector includes one or more envelope proteins from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

In some embodiments, the pseudotyped viral vector includes a VSV-G envelope protein.

In another aspect, the invention features a method of treating a cancer in a subject by identifying a sequence variant of a nORF and a cancer associated therewith, wherein the sequence of the nORF is distinct from a cORF of a gene. The nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has decreased expression relative to the nORF. The method further includes providing a protein encoded by the nORF to the subject treat the cancer.

In another aspect, the invention features a method of treating a cancer in a subject by providing a protein encoded by a nORF to the subject. The subject may have previously been identified with a sequence variant of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has decreased expression relative to the nORF.

In some embodiments of either of the foregoing aspects, the method includes restoring the encoded protein product of the WT nORF without the sequence variant. The method may include, e.g., providing the protein product or a polynucleotide encoding the protein product. The method may include, e.g., providing a vector (e.g., a viral vector) including the polynucleotide encoding the protein product.

In some embodiments, the viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include, for example one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be, for example, a lentiviral vector.

In some embodiments, the pseudotyped viral vector includes one or more envelope proteins from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

In some embodiments, the pseudotyped viral vector includes a VSV-G envelope protein.

In some embodiments of any of the above aspects, the encoded protein product of the nORF is less than about 100 amino acids.

In some embodiments, the method further includes performing a statistical analysis between the variant in the nORF and the cancer. The statistical analysis may measure a positive or negative association between the variant in the nORF and the cancer.

In some embodiments, the cancer is stomach adenocarcinoma. In some embodiments, the cancer is lung adenocarcinoma.

In some embodiments, the nORF has at least 80%, 85%, 90%, 95%, 97%, or 99% identity to SEO ID NO: 1. For example, the nORF may have the sequence of SEQ ID NO: 1. In some embodiments, the nORF has at least 80%, 85%, 90%, 95%, 97%, or 99% identity to SEQ ID NO: 2. For example, the nORF may have the sequence of SEQ ID NO: 2.

In some embodiments, the nORF is not HOXB-AS3.

In some embodiments, the cancer is not colorectal cancer.

In some embodiments, the nORF is not PINT87aa (LINC-PINT).

In some embodiments, the cancer is not glioblastoma.

In some embodiments, the small molecule is

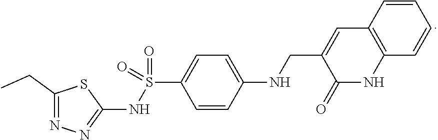 [SMILES: CCc1nnc(NS(=O)(=O)c2ccc(NCc3cc4ccccc4[nH]c3=O)cc2)s1]

In some embodiments, the small molecule is

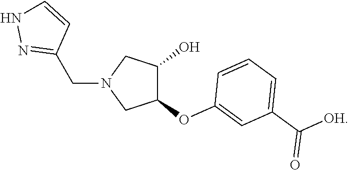 [SMILES: O=C(O)c1cccc(O[C@H]2CN(Cc3cc[nH]n3)C[C@@H]2O)c1]

In some embodiments, the small molecule is

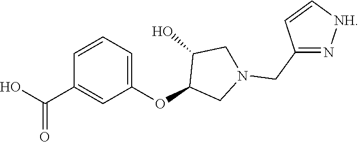 [SMILES: O=C(O)c1cccc(O[C@@H]2CN(Cc3cc[nH]n3)C[C@H]2O)c1]

### Definitions

As used herein, a “novel open reading frame” or “nORF” refers to an open reading frame that is transcribed in a cell and consists of a sequence that is distinct from a canonical open reading frame (cORF) transcribed from a gene. The nORF may be present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with a cORF or the gene. The nORF may be any unannotated genetic sequence that is transcribed in a cell.

As used herein, a “canonical open reading frame” or “cORF” refers to an open reading frame that is transcribed in a cell and its associated genetic elements, including the 5′ UTR, the 3′ UTR, the intronic regions, the exonic regions, and the intergenic regions flanking the gene comprising the cORF. A cORF includes either the primary open reading frame that is expressed from a gene, the most abundantly expressed open reading frame expressed from a gene, or an ORF that is annotated in a publicly available database as the primary and/or most abundantly expressed open reading frame from a gene.

### BRIEF DESCRIPTION OF THE DRAWINGS

FIG. 1 is a graph showing nORFs that are dysregulated in cancer. Analysis of Xena's TCGA-TARGET-GTEx dataset to study the expression of the 14 probable ‘cancer markers’ which are expressed differentially in 19 cancers. These 14 markers are non-protein-coding transcripts that translate low-noise nORFs in 11 cell lines as observed from analyzing the ribo-ORF datasets from RPFdb. Shown are transcripts that are not expressed in both the tumor and matched normal samples; transcripts that are not expressed only in tumor samples; transcripts that are expressed only in tumor samples; no differential expression of transcript between the tumor and normal samples; and differential expression of transcript between the tumor and normal samples (a transcript is defined to be expressed if it has non-zero expression in at least 25% of the samples).

FIG. 2 is a set of structures showing compounds 8462, 1491, and 1355 in complex with the predicted structure of nORF ENST00000427352.1.

### DETAILED DESCRIPTION

Described herein are methods of diagnosing and treating a cancer associated with a genetic variant. Many cancers are caused by a seemingly benign mutation, e.g., in a gene, that is associated with the cancer. However, it was previously unclear how certain benign genetic variants contribute to cancer pathology. The present invention is premised, in part, upon the discovery that certain genetic variants are also present in a novel open reading frame (nORF) that is distinct from a canonical open reading frame (cORF) of the gene. In these instances, the genetic variant imparts a deleterious effect on the nORF, with or without substantially impacting the protein encoded by the cORF. In particular, the present invention features methods of treating cancer associated with a variant nORF in which the mutation causes differential expression (e.g., increased or decreased expression) of the nORF. When the mutation causes increased or decreased expression, the gene product encoded by the variant nORF is increased or decreased as compared to the WT nORF. However, the variant may have no substantial effect on the cORF as the mutation may be conservative or silent to the gene or the protein encoded by the cORF, if the mutation is present within the cORF. The methods of diagnosis and treatment are described in more detail below.

### Methods of Diagnosis

Genetic testing offers one avenue by which a patient may be diagnosed as having or is at risk of developing a particular cancer. For example, a genetic analysis can be used to determine whether a patient has a mutation in an endogenous gene associated with a cancer. The mutation may be present in any region of the gene, such as within the cORF, a 5′ untranslated region (UTR) of the cORF, a 3′ UTR of the cORF, an intronic region of the cORF, or an intergenic region of the cORF, The mutation is also present in an nORF. The nORF may be present within an overlapping region of the cORF in an alternate reading frame, a 5′ UTR of the cORF, a 3′ UTR of the cORF, an intronic region of the cORF, or an intergenic region of the cORF. The nORF may be present in a region that is not associated with the cORF or the gene.

Exemplary genetic tests that can be used to determine whether a patient has such a mutation in the gene or the nORF include polymerase chain reaction (PCR) methods known in the art, such as DNA and RNA sequencing. In some embodiments, the subject is identified as having a certain mutation in a gene, and this mutation may be annotated in a publicly available database as being associated with a certain cancer. nORF sequences may be identified de novo, e.g., using computational or statistical methods. Furthermore, nORF sequences may be identified from publicly available databases in genomic sequences in which the nORF was not previously identified and/or annotated as a sequence that was expressed and/or translated.

nORF sequences may be identified as being linked to a particular cancer by using a statistical analysis between the variant in the nORF and the cancer. The statistical analysis may, for example, measure a positive or negative association between the variant in the nORF and the cancer (see, e.g., Example 1). To examine the functional importance of a nORF separately from a canonical coding sequence, variant frequencies from datasets, such as the Genome Aggregation Database, may be used.

### Methods of Treatment

The invention features methods of treating a subject having a cancer associated variant in an nORF that causes differential expression (e.g., increased or decreased expression). The variant nORF may exhibit an increase (e.g. by at least 5%, 10%, 15%, 20%, 25%, 30%, 40%, 50%, 60%, 70%, 80%, 90%, 100%, 200%, 300%, 400%, 500%, or more) in expression, e.g., as compared to the WT nORF or as compared to the variant nORF in normal (e.g., noncancerous) tissue. The variant nORF may exhibit a decrease (e.g., by at least 5%, 10%, 15%, 20%, 25%, 30%, 40%, 50%, 60%, 70%, 80%, 90%, 95%, 97%, or 99%) in expression, e.g., as compared to the WT nORF or as compared to the variant nORF in normal (e.g., noncancerous) tissue. The subject may be first determined to have the variant and then may be subsequently be treated for the cancer. The subject may have previously been determined to have the variant and is then treated for the cancer. The treatment varies according to the variant nORF associated with the cancer. For example, the treatment may include an inhibitor that targets the variant nORF to decrease (e.g., by at least 5%, 10%, 15%, 20%, 25%, 30%, 40%, 50%, 60%, 70%, 80%, 90%, 95%, 97%, or 99%) expression of an upregulated variant nORF. The treatment may include an activator that targets the variant nORF to increase (e.g., by at least 5%, 10%, 15%, 20%, 25%, 30%, 40%, 50%, 60%, 70%, 80%, 90%, 100%, 200%, 300%, 400%, 500%, or more) expression of a downregulated variant nORF. Alternatively, or in addition, the treatment may include providing trio WT nORF or a protein encoded by the WT nORF without the sequence variant to restore levels of the nORF.

### Inhibitors

The methods of treatment and diagnosis described herein may include providing an inhibitor that targets the variant nORF. The inhibitor may reduce (e.g., by at least 5%, 10%, 15%, 20%, 25%, 30%, 40%, 50%, 60%, 70%, 80%, 90%, 95%, 97%, or 99%) an amount or activity of the variant nORF, such as to prevent the deleterious effect of the variant nORF. The inhibitor may target the polynucleotide containing the nORF or the protein encoded by the nORF. The inhibitor may be, for example, a small molecule, a polynucleotide, or a polypeptide. Suitable small molecules may be determined or identified, for example, by using computational analysis based on the structure of the variant nORF as determined by a protein folding algorithm. The small molecule may target any region of the variant nORF. The small molecule may target the nORF or the protein encoded by the nORF. Suitable polypeptides for reducing an activity or amount of the variant nORF include, for example, an antibody or antigen-binding fragment thereof that binds to the variant nORF (e.g., a single chain antibody or antigen-binding fragment thereof). Suitable polynucleotides that can reduce an amount or activity of the variant nORF include RNA. For example, an RNA for reducing an activity or amount of the variant nORF may be, for example, a miRNA, an antisense RNA, an shRNA, or an siRNA. The miRNA, antisense RNA, shRNA, or siRNA may target a region of RNA (e.g., variant nORF gene) to reduce expression of the variant nORF. The polynucleotide may be, e.g., an aptamer, e.g., an RNA aptamer that binds to and/or reduces an amount and/or activity of the variant nORF or the protein encoded by the variant nORF. The inhibitor may be provided directly or may be provided by a vector (e.g., a viral vector) encoding the inhibitor. The inhibitor may be formulated, e.g., in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle.

### Nucleic Acid Mediated Knockdown

Using the compositions and methods described herein, a patient with a cancer may be administered an interfering RNA molecule, a composition containing the same, or a vector encoding the same, so as to reduce or suppress the expression of a variant nORF. Exemplary interfering RNA molecules that may be used in conjunction with the compositions and methods described herein are siRNA molecules, miRNA molecules, shRNA molecules, and antisense RNA molecules, among others. In the case of siRNA molecules, the siRNA may be single stranded or double stranded, miRNA molecules, in contrast, are single-stranded molecules that form a hairpin, thereby adopting a hydrogen-bonded structure reminiscent of a nucleic acid duplex. In either case, the interfering RNA may contain an antisense or “guide” strand that anneals (e.g., by way of complementarity) to the repeat-expanded mutant RNA target. The interfering RNA may also contain a “passenger” strand that is complementary to the guide strand and, thus, may have the same nucleic acid sequence as the RNA target.

siRNA is a class of short (e.g., 20-25 nt) double-stranded non-coding RNA that operates within the RNA interference pathway. siRNA may interfere with expression of the variant nORF gene with complementary nucleotide sequences by degrading mRNA (via the Dicer and RISC pathways) after transcription, thereby preventing translation. mRNA is another short (e.g., about 22 nucleotides) non-coding RNA molecule that functions in RNA silencing and post-transcriptional regulation of gene expression. miRNAs function via base-pairing with complementary sequences within mRNA molecules, thereby leading to cleavage of the mRNA strand into two pieces and destabilization of the mRNA through shortening of its poly(A) tail. shRNA is an artificial RNA molecule with a tight hairpin turn that can be used to silence target gene expression via RNA interference. Antisense RNA are also short single stranded molecules that hybridize to a target RNA and prevent translation by occluding the translation machinery, thereby reducing expression of the target (e.g., the variant nORF).

### Antibody Mediated Knockdown

Using the compositions and methods described herein, a patient with a cancer may be provided an antibody or antigen-binding fragment thereof, a composition containing the same, a vector encoding the same, or a composition of cells containing a vector encoding the same, so as to suppress or reduce the activity of the variant nORF. In some embodiments of the compositions and methods described herein, an antibody or antigen-biding fragment thereof may be used that binds to and reduces or eliminates the activity of the variant nORF. The antibody may be, for example, monoclonal or polyclonal. In some embodiments, the antigen-binding fragment is an antibody that lacks the Fc portion, an F(ab′)₂, a Fab, an Fv, or an scFv. The antigen-binding fragment may be an scFv.

One of ordinary skill in the art will appreciate that an antibody may include four polypeptides: two identical copies of a heavy chain polypeptide and two copies of a light chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C_{L}) region. Thus, one of skill in the art would appreciate that as described herein, a vector that includes a transgene that encodes a polypeptide that is an antibody may be a single transgene that encodes a plurality of polypeptides. Also contemplated is a vector that includes a plurality of transgenes, each transgene encoding a separate polypeptide of the antibody. All variations are contemplated herein. The variable regions of each pair of light and heavy chains form the antigen binding site of an antibody. The transgene which encodes an antibody directed against the variant nORF can include one or more transgene sequences, each of which encodes one or more of the heavy and/or light chain polypeptides of an antibody. In this respect, the transgene sequence which encodes an antibody directed against the variant nORF can include a single transgene sequence that encodes the two heavy chain polypeptides and the two light chain polypeptides of an antibody. Alternatively, the transgene sequence which encodes an antibody directed against the variant nORF can include a first transgene sequence that encodes both heavy chain polypeptides of an antibody, and a second transgene sequence that encodes both light chain polypeptides of an antibody. In yet another embodiment, the transgene sequence which encodes an antibody can include a first transgene sequence encoding a first heavy chain polypeptide of an antibody, a second transgene sequence encoding a second heavy chain polypeptide of an antibody, a third transgene sequence encoding a first light chain polypeptide of an antibody, and a fourth transgene sequence encoding a second light chain polypeptide of an antibody.

In some embodiments, the transgene that encodes the antibody includes a single open reading frame encoding a heavy chain and a light chain, and each chain is separated by a protease cleavage site.

In some embodiments, the transgene encodes a single open reading frame encoding both heavy chains and both light chains, and each chain is separate by protease cleavage site.

In some embodiments, full-length antibody expression can be achieved from a single transgene cassette using 2A peptides, such as foot-and-mouth disease virus (FMDV) equine rhinitis A, porcine teschovirus-1, and Thosea asigna virus 2A peptides, which are used to link two or more genes and allow the translated polypeptide to be self-cleaved into individual polypeptide chains (e.g., heavy chain and light chain, or two heavy chains and two light chains). Thus, in some embodiments, the transgene encodes a 2A peptide in between the heavy and light chains, optionally with a flexible linker flanking the 2A peptide (e.g., GSG linker). The transgene may further include one or more engineered cleavage sequences, e.g., a furin cleavage sequence to remove the 2A peptide residues attached to the heavy chain or light chain. Exemplary 2A peptides are described, e.g., in Cling et al MAbs 7: 403-412, 201f5, and Lin et al. Front. Plant Sci. 9:1379, 2018, the disclosures of which are hereby incorporated by reference in their entirety.

In some embodiments, the antibody is a single-chain antibody or antigen-binding fragment thereof expressed from a single transgene.

### Activators

The methods of treatment and diagnosis described herein may include providing an activator that targets the variant nORF. The activator may increase (e.g., by at least 5%, 10%, 15%, 20%, 25%, 30%, 40%, 50%, 60%, 70%, 80%, 90%, 100%, 200%, 300%, 400%, 500%, or more) an amount or activity of the variant nORF, such as to prevent the deleterious effect of the variant nORF. The activator may target the polynucleotide containing the nORF or the protein encoded by the nORF. The activator may be, for example, a small molecule, a polynucleotide, or a polypeptide. Suitable small molecules may be determined or identified, e.g., by using computational analysis based on the structure of the variant nORF as determined by a protein folding algorithm. The small molecule may target any region of the variant nORF. The small molecule may target the nORF or the protein encoded by the nORF, Suitable polypeptides for increasing an activity or amount of the variant nORF include, for example, an antibody or antigen-binding fragment thereof that binds to the variant nORF (e.g., a single chain antibody or antigen-binding fragment thereof). Suitable polynucleotides that can increase an amount or activity of the variant nORF include RNA. For example, an RNA for increasing an activity or amount of the variant nORF may be, for example, an antisense RNA. The antisense RNA may target a region of RNA (e.g., variant nORF gene) upstream of the primary nORF open reading frame to reduce expression of the upstream nORFs, thereby dedicating the translation machinery to the primary nORF in order to increase expression of the variant primary nORF. The polynucleotide may be an aptamer, e.g., an RNA aptamer that binds to and/or increases an amount and/or activity of the variant nORF or the protein encoded by the variant nORF. The activator may be provided directly or may be provided by a vector (e.g., a viral vector) encoding the activator. The activator may be formulated, e.g., in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle.

nORF Replacement

The present invention also features methods of treating a cancer by administering or providing a WT nORF or a protein encoded by the WT nORF. The therapy may restore the encoded protein product of the WT nORF without the sequence variant, such as to replace the WT nORF that is no longer present due to the mutation. The therapy may include, for example, providing the protein product or a polynucleotide encoding the protein product. The method may include providing a vector (e.g., a viral vector) that encodes the protein product. Alternatively, the protein encoded by the nORF may be administered directly, e.g., as an enzyme replacement therapy. The WI nORF or a polynucleotide encoding the WT nORF (e.g., a vector, e.g., a viral vector) may be formulated, e.g., in a pharmaceutical composition containing a pharmaceutically acceptable carrier. The composition can be administered by any suitable method known in the art to the skilled artisan. The composition may be formulated in a virus or a virus-like particle.

In some embodiments, the length of the WT nORF is less than about 100 amino acids (e.g., from about 50 to 100, 50 to 90, 50 to 80, 60 to 90, 60 to 80, 70 to 100, 70 to 90, 70 to 80, 80 to 100, or 90 to 100 amino acids).

### Viral Vectors for Expression

Viral genomes provide a rich source of vectors that can be used for the efficient delivery of exogenous genes into a mammalian cell. The gene to be delivered may include, for example, an activator or inhibitor that targets a variant nORF, such as an RNA (e.g., an aptamer, a miRNA, an antisense RNA, an shRNA, or an siRNA). Alternatively, the gene to be delivered may include the WT nORF for replacement. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., an adeno-associated viral (AAV) vector), coronavirus, negative strand RNA viruses such as orthomyxovirus influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus. Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods described herein may be, for example, a retroviral vector. One type of retroviral vector that may be used in the methods and compositions described herein is a lentiviral vector. Lentiviral vectors (LVs), a subset of retroviruses, transduce, a wide range of dividing and non-dividing cell types with high efficiency, conferring stable, long-term expression of the transgene encoding the polypeptide or RNA. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the agent of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include, for example, one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Thor.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

The viral vectors (e.g., retroviral vectors, e.g., lentiviral vectors) may include a promoter operably coupled to the transgene encoding the polypeptide or the polynucleotide encoding the RNA to control expression. The promoter may be, for example, a ubiquitous promoter. Alternatively, the promoter may be a tissue specific promoter, such as a myeloid cell-specific or hepatocyte-specific promoter. Suitable promoters that may be used with the compositions described herein include CD11b promoter, sp146/p47 promoter, CD68 promoter, sp146/gp9 promoter, elongation factor 1α (EF1α) promoter, EF1α short form (EFS) promoter, phosphoglycerate, kinase (PGK) promoter, α-globin promoter, and β-globin promoter. Other promoters that may be used include, e.g., DC172 promoter, human serum albumin promoter, alpha1 antitrypsin promoter, thyroxine binding globulin promoter. The DC172 promoter is described in Jacob, et al. Gene Ther. 15:594-603, 2008, hereby incorporated by reference in its entirety.

The viral vectors (e.g., retroviral vectors, e.g., lentiviral vectors) may include an enhancer operably coupled to the transgene encoding the polypeptide or the polynucleotide encoding the RNA to control expression. The enhancer may include a β-globin locus control region (βLCR).

Methods of Measuring nORF Gene Expression

Preferably, the compositions and methods of the disclosure are used to facilitate expression of a WT nORF at physiologically normal levels in a patient (e.g., a human patient), decrease expression of an upregulated nORF, or increase expression of a downregulated nORF. The therapeutic agents of the disclosure, for example, may reduce the variant nORF expression in a human subject. For example, the therapeutic agents of the disclosure may reduce variant nORF expression e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, 98%, or 99%. Alternatively, the therapeutic agents of the disclosure, for example, may increase the variant nORF expression in a human subject. For example, the therapeutic agents of the disclosure may increase variant nORF expression, e.g., by at least 5%, 10%, 15%, 20%, 25%, 30%, 40%, 50%, 60%, 70%, 80%, 90%, 100%, 200%, 300%, 400%, 500%, or more.

The expression level of the nORF expressed in a patient can be ascertained, for example, by evaluating the concentration or relative abundance of mRNA transcripts derived from transcription of the nORF. Additionally, or alternatively, expression can be determined by evaluating the concentration or relative abundance of the nORF following transcription and/or translation of an inhibitor that decreases an amount of the variant nORF. Protein concentrations can also be assessed using functional assays, such as MDP detection assays. Expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### Nucleic Acid Detection

Nucleic acid-based methods for determining expression (e.g., of an RNA inhibitor or an RNA encoding the WT nORF) detection that may be used in conjunction with the compositions and methods described herein include, for example, imaging-based techniques (e.g., Northern blotting or Southern blotting). Such techniques may be performed using cells obtained from a patient following administration of the polynucleotide encoding the agent. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, NY 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Detection techniques that may be used in conjunction with the compositions and methods described herein to evaluate nORF expression further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, expression at the mRNA level may be determined, e.g., using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Expression levels of the nORF may be determined, for example, using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified, for example, according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of the nORF or RNA in a target cell following delivery to a patient. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Held et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of the nORF can additionally be determined by measuring the concentration or relative abundance of a corresponding protein product (e.g., the WT nORF or the variant nORF). Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize expression of the nORF in a cell from a patient (e.g., a human patient) following delivery of the transgene encoding the nORF. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Cancer

A number of cancers are known in the art that are associated with a variant. However, the present invention contemplates treatment of a cancer in which the variant may be benign in the associated canonical ORF of the gene but has a deleterious effect in the nORF. The skilled artisan practicing the invention can identify the variant in the nORF using the methods described herein. Alternatively, the skilled artisan could identify a benign variant in a cORF and determine whether that cORF contains an associated nORF. The skilled person may further determine whether the variant is present within the nORF and whether this variant causes increased or decreased expression of the variant nORF.

The method may, for example reduce the size (e.g., by 5%, 10%, 15%, 20%, 25%, 30%, 40%, 50%, 60%, 70%, 80%, 90%, 95%, 97%, or 99%) of a tumor. The method may, for example decrease or slow (e.g., by at least 5%, 10%, 15%, 20%, 25%, 30%, 40%, 50%, 60%, 70%, 80%, 90%, 95%, 97%, or 99%) the progression of cancer. The method may, for example, decrease (e.g., by at least 5%, 10%, 15%, 20%, 25%, 30%, 40%, 50%, 60%, 70%, 80%, 90%, 95%, 97%, or 99%) the risk of developing cancer. The method may decrease (e.g., by at least 5%, 10%, 15%, 20%, 25%, 30%, 40%, 50%, 60%, 70%, 80%, 90%, 95%, 97%, or 99%) the risk of developing cancer.

In some embodiments, the cancer is stomach adenocarcinoma. In some embodiments, the cancer is lung adenocarcinoma.

In some embodiments, the nORF has least 80%, 85%, 90%, 95%, 97%, or 99% identity to SEQ ID NO: 1 or 2. For example, the nORF may have the sequence of SEQ ID NO: 1 or 2.

In some embodiments, the nORF is not HOXB-AS3.

In some embodiments, the cancer is not colorectal cancer.

In some embodiments, the nORF is not PINT87aa (LINC-PINT).

In some embodiments, the cancer is not glioblastoma.

In some embodiments, the small molecule is:

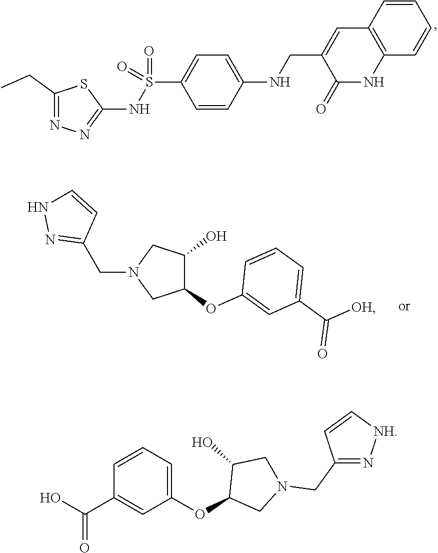 [SMILES: CCc1nnc(NS(=O)(=O)c2ccc(NCc3cc4ccccc4[nH]c3=O)cc2)s1 ; O=C(O)c1cccc(O[C@H]2CN(Cc3cc[nH]n3)C[C@@H]2O)c1 ; "or" ; O=C(O)c1cccc(O[C@@H]2CN(Cc3cc[nH]n3)C[C@H]2O)c1]

### EXAMPLES

The following examples further illustrate the invention but should not be construed as in any way limiting its scope.

### Example 1. Dysregulation of nORFs in Cancer and Screen for Inhibitors

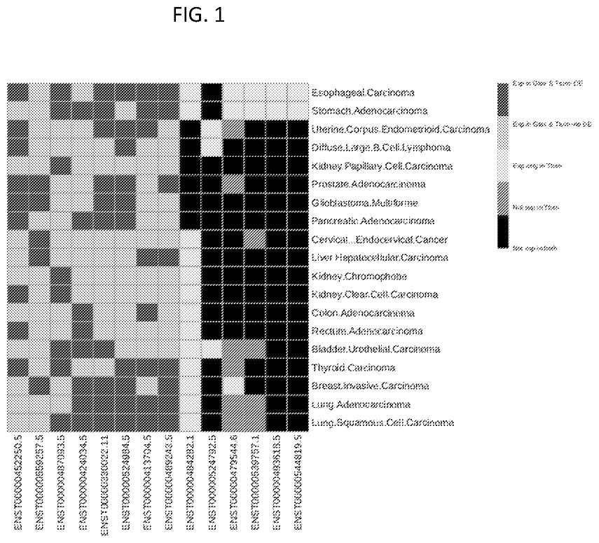

To show that novel proteins are dysregulated in cancer we identified 14 novel ORFs that are identified to be translated with ‘low-noise’ in 11 human cell lines from the ribo-ORF datasets 24. The expression of these 14 transcripts in cancer was then analyzed using the USCS Toil Recompute and found to be differentially expressed in 19 of the 33 cancer types, in spite of using a very stringent criteria for this analysis (FIG. 1). This indicates that they might be dysregulated and have some role in cancer.

Interestingly, ENST00000484282.1 is expressed only in tumor samples and not in their matched healthy tissues across almost 70% of the TCGA cancer types (FIG. 1). Encoded by the DOP1A gene (DOP1 leucine zipper like protein A; ENSG00000083097), ENST00000484282.1 is annotated as a processed transcript, and therefore, by definition does not contain an ORF. Analysis with the RPFdbv2.0 datasets showed that this transcript translates a ‘low noise’ ORF with an ATG start codon in all the 11 human cell lines analyzed (many of which are cancer cell lines). Thus, this transcript, which is expressed only in tumor samples may potentially express nORF with some specific function in tumors.

Additionally, to investigate whether novel proteins dysregulated in cancer can be used as therapeutic targets, we predicted structure of the human ortholog of a nORF, mPLsORF0000447155, identified in our B and T cell study, translated from ENST00000427352.1, and identified to be expressed only in the tumor samples of Stomach adenocarcinoma and Esophageal carcinoma and with two noncoding mutations mapped to them (COSN19210254; COSN8491742;) of which; COSN8491742 is identified in only lung samples (Table 1). We then screened for highest scoring ligands from the asinex library against Immuno Oncology (8462 compounds), targeted Oncology (1491 compounds), Signaling pathway inhibitors (1355 compounds). We also identified the top scoring ligands for the above categories. These results reveal that novel proteins are not only dysregulated in cancer but can also be used for diagnostic and for therapeutic purposes.

TABLE 1
List of cosmic mutations

| | | | nORF |
|---|---|---|---|
| | | Effect | secondary |
| Mutation ID(s) | Change | on nORF | structure |
| COSN19210254 | 115553735 T > A | K5 > K | C |
| #COSN8491742 | 115553987 C > A | A89 > T | C |

Table 1 shows the list of cosmic mutation IDs for COSN19210254 and COSN8491742 along with the nucleic acid change, predicted amino acid change according to the standard amino acid code, and the predicted secondary structure of the protein at that position. C=Coil.
Mouse nORFs Structure Prediction and Mutation Mapping

Human ortholog transcript of one mouse nORF that is translated in mouse B and T cells was identified, its structure was predicted, and inhibitors were screened against it. The details are as follows.

Human ortholog of mPLsORF0000447155 nORF was identified using tblastn+liftover (e-value: 4.00E-19, length: 90, pident: 91.11, mismatch: 8), and it maps to a genomic location of a human transcript ENST00000427352.1: chr5:115553723-115553992:—(GRCh37). This transcript ‘ENST00000427352.1’, annotated is ‘processed_pseudogene’, is expressed only in the tumor samples of Stomach adenocarcinoma, Esophageal carcinoma, acute myeloid leukemia and is expressed only in the normal samples of Testicular germ cell tumor. We call a transcript expressed in particular condition if it has non-zero expression in more than 10% of the samples. We mapped two cosmic noncoding mutations to this transcript. Structure of the human nORF was predicted using Evfold pipeline with the following parameters: Bit score=0.2, seqlen=90, N_eff/L=3.85, number of effective sequences=342, number of sequences in alignment (num_seqs)=1063, perc_cov=0.944.

Inhibitor Screens for the Two nORFs Identified to be Disrupted in Cancers

Structure predicted from ENST00000427352.1 (human ortholog of mPLsORF0000447155 nORF) was chosen for drug screening study. Briefly, structure based virtual screening analysis was performed using Virtual screening workflow of Schrödinger software suite. First in the protein preparation step, the structure was minimized using protein preparation wizard in maestro 12.1 (Schrodinger) applying force field OPLS3 with default parameters. Next, the active sites were predicted using SiteMap (Schrodinger) and CastP. The grid was generated at all the active site residues of the topmost scoring pocket identified by the two tools.

mPLsORF0000447155:
(SEQ ID NO: 1)
MPKRKAEGDAKGDKTKVKDEPQRRSARLSAKPAPPKPEPKPKKAPAKKGE
KVPKGKKGKADAGKDANNPAENGDAKTDQAQKAEGAGDAK
Peptide sequence of the product translated from
ENST00000427352.1:
(SEQ ID NO: 2)
MPKRKAEGDAKGDKAKVKDEPQRRSARLSAKPASPKPEPRPKKAPAKKGE
KVPKGRKGKADAGKEGNNPAENGDVKTDQAQKAEGAGGAK.

TABLE 2
Predicted active Site Residues used in docking

| Software | Residues | Residues selected for docking |
|---|---|---|
| Castp | Pro2, Ala6, Glu7, Gly8, Lys11 | Pro2, Ala6, Glu7, Gly8, Lys11, |
| | Gly12, Lys14, Gln22, Arg23, | Gly12, Asp13, Thr15, |
| | Arg24 | Gln22, Arg23, Arg24 |
| | Ala30, Pro32, ARG40, Pro41, | Ala30, Pro32, ARG40, Pro41, |
| | Lys51, Arg56, Lys57, Ala60, | Lys51, Arg56, lys57, Ala60, |
| | Asn72, Gly73, Val75, Lys76, | Asn72, Gly73, Val75, Lys76, |
| | Thr77, Ala80, Gln81 | Thr77, Ala80, Gln81 |
| SiteMap | Pro2, Glu7, Gly8, Asp13, Thr15, | |
| | Ala60, Ala80, Gln81 | |

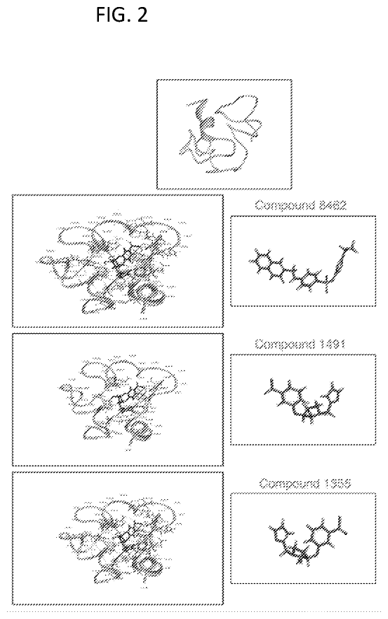

Evfold predicted structure of the translated product of ENST00000427352.1 with the marked active site residues. The virtual screening involved the following three stages: 1. HTVS (High throughput virtual screening), 2. SP (Standard Precision), and 3. XP (Extra Precision) docking. The small molecules of the following three libraries obtained from Asinex library was used for docking: Immuno oncology (11346) compounds (asinex.com/wp-content/uploads/2017/01/2016-11-Asinex-Immuno-Oncology-11346.zip), targeted oncology (6728) compounds (asinex.com/wp-content/uploads/2016/11/2016-11-Asinex-Targeted-Oncoiogy-6728.zip) and signal pathway inhibitors (5923) (hasinex.com/wp-content/uploads/2017/01/2016-11-Asinex-Signal-Pathway-Inhibitors-5923.zip). The 2D SDP format of all the compounds structures in these libraries were converted into 3D format using Schrodinger's LigPrep module with OPLS3 Force Field (FIG. 2). A three-step docking methodology was used—Glide HTVS, SP and XP. Listed below are the details of the predicted best hit compounds searched from the three asinex libraries.

TABLE 3
ImmunoOncology library

| Compound ID | Docking Score |
|---|---|
| 8462 | −7.011 |
| 11233 | −6.436 |
| 10977 | −6.029 |
| 11189 | −5.996 |
| 10976 | −5.678 |
| 11212 | −5.473 |
| 4965 | −5.187 |
| 8554 | −4.966 |
| 10035 | −4.774 |
| 9994 | −4.698 |
| 11188 | −4.689 |
| 10516 | −4.433 |
| 9987 | −4.399 |
| 10922 | −4.390 |
| 10413 | −4.387 |
| 10547 | −4.263 |
| 11232 | −4.214 |

We identified the structure of the predicted best hit molecule (compound 8462) and its complex with the target protein. The Interacting residues are Asn 72, Arg 40 and Met 1. Tabulated below are the MM-GBSA binding energies, which estimate relative binding affinities for the few best hit compounds.

TABLE 4
MMGBSA Energy Calculation

| Top Compounds | Binding energy(Kcal/mol) |
|---|---|
| 8462 | −38.68 |
| 11233 | −45.76 |
| 10977 | −45.53 |
| 11189 | −33.22 |

TABLE 5
Docking scores

| Compound Id | Docking Score |
|---|---|
| 1491 | −7.114 |
| 139 | −6.883 |
| 1479 | −6.739 |
| 700 | −6.662 |
| 140 | −6.496 |
| 3256 | −6.268 |
| 6649 | −5.997 |
| 4095 | −5.987 |
| 1581 | −5.974 |
| 3104 | −5.959 |
| 4093 | −5.952 |

Structure of the predicted best hit molecule (compound 1491) (bottom) and its complex with the target protein (top). The interacting residues are Gly8, Arg40, Lys51, Lys57 and Gln81.

Tabulated below are the MMGBSA binding energies and docking scores for the few top hits.

TABLE 6
MMGBSA energy calculation

| Top Compounds | Binding Energy (Kcal/mol) |
|---|---|
| 1491 | −44.31 |
| 139 | −35.59 |
| 1479 | −43.03 |
| 700 | −38.03 |
| 140 | −47.83 |

TABLE 7
Docking scores

| Compound Id | Docking Score |
|---|---|
| 1355 | −7.238 |
| 129 | −6.883 |
| 687 | −6.662 |
| 1347 | −6.631 |

We predicted the structure of the predicted best hit molecule (compound 1355) and its complex with the target protein. The interacting residues are Gly8, ARG 40 and Gln81

TABLE 8
MMGBSA energy calculations for few top compounds

| Top Compounds | Binding energy(Kcal/mol) |
|---|---|
| 1355 | −7.238 |
| 129 | −6.883 |
| 687 | −6.662 |
| 1347 | −6.631 |

The structures of compounds 8462, 1491, and 1355 are as follows:

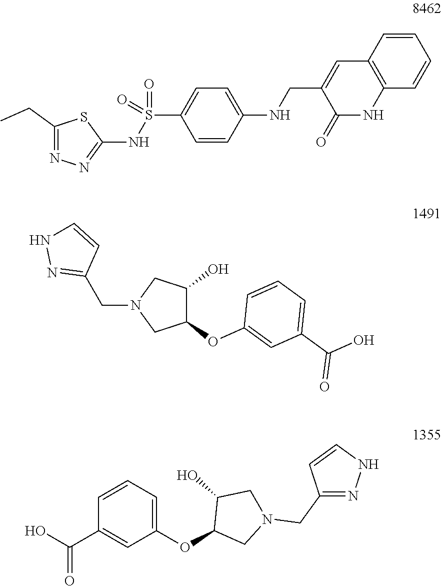 [SMILES: CCc1nnc(NS(=O)(=O)c2ccc(NCc3cc4ccccc4[nH]c3=O)cc2)s1 ; O=C(O)c1cccc(O[C@H]2CN(Cc3cc[nH]n3)C[C@@H]2O)c1 ; O=C(O)c1cccc(O[C@@H]2CN(Cc3cc[nH]n3)C[C@H]2O)c1]

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating a cancer in a subject comprising:
(a) identifying a sequence variant of a novel open reading frame (nORF) and a cancer associated therewith, wherein the sequence of the nORF is distinct from a canonical open reading frame (cORF) of a gene, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene, and wherein the variant nORF has increased expression relative to the nORF; and
(b) administering to the subject an inhibitor that reduces expression of the variant nORF to treat the cancer.

2. A method of treating a cancer in a subject comprising administering to the subject an inhibitor that reduces expression of a variant nORF; wherein the subject has previously been identified with a sequence variant of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has increased expression relative to the nORF.

3. The method of any one of claim 1 or 2, wherein the inhibitor comprises a small molecule, a polynucleotide, or a polypeptide.

4. The method of claim 3, wherein the polynucleotide comprises a miRNA, an antisense RNA, an shRNA, or an siRNA.

5. The method of claim 3, wherein the polypeptide comprises an antibody or antigen-binding fragment thereof.

6. The method of claim 5, wherein the antigen-binding fragment thereof is an scFv.

7. The method of any one of claims 3 to 6, wherein the inhibitor is encoded by a vector.

8. The method of claim 7, wherein the vector is a viral vector.

9. The method of claim 8, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

10. The method of claim 9, wherein the parvovirus viral vector is an adeno-associated virus (AAV) vector.

11. The method of claim 10, wherein the viral vector is a Retroviridae family viral vector.

12. The method of claim 11, wherein the Retroviridae family viral vector is a lentiviral vector.

13. The method of claim 11, wherein the Retroviridae family viral vector is an alpharetroviral vector or a gammaretroviral vector.

14. The method of any one of claims 10 to 13, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

15. The method of any one of claims 10 to 14, wherein the viral vector is a pseudotyped viral vector.

16. The method of claim 15, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

17. The method of claim 16, wherein the pseudotyped viral vector is a lentiviral vector.

18. The method of any one of claims 15 to 17, wherein the pseudotyped viral vector comprises one or more envelope proteins from a virus selected from vesicular stomatitis virus (VSV), RD1 14 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

19. The method of claim 18, wherein the pseudotyped viral vector comprises a VSV-G envelope protein.

20. A method of treating a cancer in a subject comprising:
(a) identifying a sequence variant of a nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has decreased expression relative to the nORF; and
(b) administering to the subject an activator that increases expression of variant nORF to treat the cancer.

21. A method of treating a cancer in a subject comprising administering to the subject an activator that increases expression of a variant nORF; wherein the subject has previously been identified with a sequence variant of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has decreased expression relative to the nORF.

22. The method of claim 20 or 21, wherein the activator comprises a small molecule, a polynucleotide, or a polypeptide.

23. The method of claim 22, wherein the polynucleotide comprises an antisense RNA.

24. The method of claim 22, wherein the polypeptide comprises an antibody or antigen-binding fragment thereof.

25. The method of claim 24, wherein the antigen-binding fragment thereof is an scFv.

26. The method of any one of claims 20 to 25, wherein the activator is encoded by a vector.

27. The method of claim 26, wherein the vector is a viral vector.

28. A method of treating a cancer in a subject comprising:
(a) identifying a sequence variant of a nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has decreased expression relative to the nORF; and
(b) providing a protein encoded by the nORF to the subject treat the cancer.

29. A method of treating a cancer in a subject comprising providing a protein encoded by a nORF to the subject; wherein the subject has previously been identified with a sequence variant of the nORF and a cancer associated therewith, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the variant nORF has decreased expression relative to the nORF.

30. The method of claim 28 or 29. wherein the method comprises restoring the encoded protein product of the WT nORF without the sequence variant.

31. The method of claim 30, wherein the therapy comprises providing the protein product or a polynucleotide encoding the protein product.

32. The method of claim 31, wherein the method comprises providing a vector comprising the polynucleotide encoding the protein product.

33. The method of claim 32, wherein the vector is a viral vector.

34. The method of claim 33, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

35. The method of claim 34, wherein the parvovirus viral vector is an AAV vector.

36. The method of claim 35, wherein the viral vector is a Retroviridae family viral vector.

37. The method of claim 36, wherein the Retroviridae family viral vector is a lentiviral vector.

38. The method of claim 36, wherein the Retroviridae family viral vector is an alpharetroviral vector or a gammaretroviral vector.

39. The method of any one of claims 34 to 37, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

40. The method of any one of claims 33 to 39, wherein the viral vector is a pseudotyped viral vector.

41. The method of claim 40, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

42. The method of claim 41, wherein the pseudotyped viral vector is a lentiviral vector.

43. The method of any one of claims 39 to 42, wherein the pseudotyped viral vector comprises one or more envelope proteins from a virus selected from vesicular stomatitis virus VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

44. The method of claim 43, wherein the pseudotyped viral vector comprises a VSV-G envelope protein.

45. The method of any one of claims 1 to 44, wherein the encoded protein product of the nORF is less than about 100 amino acids.

46. The method of any one of claims 1 to 45, further comprising performing a statistical analysis between the variant in the nORF and the cancer.

47. The method of claim 46, wherein the statistical analysis measures a positive or negative association between the variant in the nORF and the cancer.

48. The method of any one of claims 1 to 47, wherein the cancer is stomach adenocarcinoma.

49. The method of any one of claims 1 to 47, wherein the cancer is lung adenocarcinoma.

50. The method of claim 48 or 49, wherein the nORF has at least 80%, 85%, 90%, 95%, 97%, or 99% identity to SEQ ID NO: 1 or 2.

51. The method of claim 50, wherein the nORF has the sequence of SEQ ID NO: 1 or 2.

52. The method of any one of claim 3-19 or 22-27, wherein the small molecule is: [chemical structure]
